(19) United States
(12) Patent Application Publication
(10) Pub. No.: US 2014/0371619 A1
(43) Pub. Date: Dec. 18, 2014

(54) GAS SENSOR WITH HEATER

(71) Applicant: Steven A. Rodriguez, Seattle, WA (US)

(72) Inventor: Steven A. Rodriguez, Seattle, WA (US)

(73) Assignee: GM Nameplate, Inc., Seattle, WA (US)

(21) Appl. No.: 14/304,359

(22) Filed: Jun. 13, 2014

Related U.S. Application Data
(60) Provisional application No. 61/834,647, filed on Jun. 13, 2013.

(51) Int. Cl. A61B 5/08

(52) U.S. Cl. CPC A61B 5/082
USPC 600/532

(57) ABSTRACT

A first embodiment of a disclosed breath analyzer detects a particular breath component in a breath sample. The analyzer includes a housing defining an interior cavity and having an inlet aperture for receiving the breath sample and an outlet aperture. A sensor is disposed within the cavity for sensing the component of the breath sample. An anemometer circuit is associated with the sensor and measures a rate of flow of the breath sample within the housing. The analyzer further includes a controller operatively connected to the sensor to receive breath component information sensed by the sensor.

### CROSS-REFERENCE TO RELATED APPLICATION

This application claims the benefit of U.S. Provisional Application No. 61/834,647, filed Jun. 13, 2013, the entire disclosure of which is hereby incorporated by reference herein.

### BACKGROUND

Exhaled human breath typically consists of approximately 78% nitrogen, 15-18% oxygen, 4-6% carbon dioxide, and 5% water. The remaining small fraction of exhaled breath generally consists of trace levels of more than 1000 volatile organic compounds (VOCs) with concentrations ranging from parts per trillion (pptv) to parts per million (ppmv).

Acetone is a VOC in exhaled human breath that can indicate various health conditions such as diabetes, heart disease, epilepsy, and others. For example, a person with diabetes who is in a state of ketosis will have an increased breath concentration of acetone resulting from the body's production of ketone bodies. Acetone is also produced by ketosis resulting from a restricted calorie weight loss and/or exercise program. This acetone production is the result of metabolism of fat. Hence, a breath acetone content measurement can be used as an indication of a medical condition or of fat burning during a diet and/or program to show the effectiveness of the program. These examples should be considered non-limiting in that the present disclosure can be directed to any situation in which breath acetone levels are to be detected and/or monitored.

The present disclosure is directed to an acetone sensor useful for detecting various health conditions and/or for monitoring the efficacy of diet and exercise programs. The acetone level for diet and exercise is lower than that caused by diabetes. Accordingly, a more sensitive sensor is required to monitor increased acetone levels caused by diet and exercise. Thus, there is a need for an acetone sensor capable of detecting acetone levels corresponding to diet and exercise induced ketosis.

### SUMMARY

A first embodiment of a disclosed breath analyzer detects a particular breath component in a breath sample. The analyzer includes a housing defining an interior cavity and having an inlet aperture for receiving the breath sample and an outlet aperture. A sensor is disposed within the cavity for sensing the component of the breath sample. An anemometer circuit is associated with the sensor and measures a rate of flow of the breath sample within the housing. The analyzer further includes a controller operatively connected to the sensor to receive breath component information sensed by the sensor.

A second disclosed embodiment of a breath analyzer detects a breath component in a breath sample. The breath analyzer includes a housing that defines an interior cavity and includes an inlet aperture for receiving the breath sample and an outlet aperture. A metal oxide sensor is disposed within the cavity for sensing the component of the breath sample. The analyzer further includes a temperature control system integrally formed with the sensor. The temperature control system has a metal resistor with a positive temperature coefficient. The metal resistor is configured to heat the sensor to a predetermined temperature and to sense the temperature of the sensor. The metal resistor is integrally formed with a closed-loop control that selectively controls the metal resistor. The closed-loop control circuit is configured to measure a rate of flow of the breath sample within the housing. A controller operatively connected to the sensor to receive breath component information sensed by the sensor.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a cross-sectional side view of an acetone sensing device in accordance with the present disclosure;

FIG. 2 shows a cross-sectional view of a first exemplary embodiment of a sensor assembly of the acetone sensing device of FIG. 1;

FIG. 3 shows a cross-sectional view of a second exemplary embodiment of a sensor assembly of the acetone sensing device of FIG. 1;

FIG. 4 shows a schematic diagram of a first exemplary embodiment of a temperature control system of the sensor assembly of FIG. 2; and

FIG. 5 shows a schematic diagram of a second exemplary embodiment of a temperature control system of the sensor assembly of FIG. 2.

### DETAILED DESCRIPTION

This present disclosure relates to a device for detecting the concentration of a particular breath component, such as acetone, using a metal oxide sensor in combination with a temperature control system. The temperature control system uses a closed loop control both to sense the sensor operating temperature and to heat the sensor as necessary to achieve the desired sensor operating temperature. Metal oxide based gas sensors typically require operation at relatively high temperatures (e.g. 300 C). The sensitivity of the sensor to a given gas is often highly dependent on the sensor temperature. Therefore the ability to directly monitor and control sensor temperature is advantageous. Accurate thermal control becomes especially critical when attempting to detect gases that are present at very low concentrations such as with the acetone vapor found in breath as a result of diet and exercise. The present disclosure relates to the temperature control system used to achieve the desired sensor operating temperature, and the advantageous use of the heating element itself as the temperature measurement device.

While the present disclosure and exemplary embodiments are generally described with respect to devices used to detect acetone content in a breath sample, such embodiments are exemplary only and should not be considered limiting. In this regard, the described sensors can be sensors for detecting levels of gaseous breath components other than acetone, including VOCs and other gaseous compounds. Further, it will be appreciated that the sensors are not limited to sensors used for detecting components from breath samples, but can include sensors used to detect components of any other suitable gas sample.

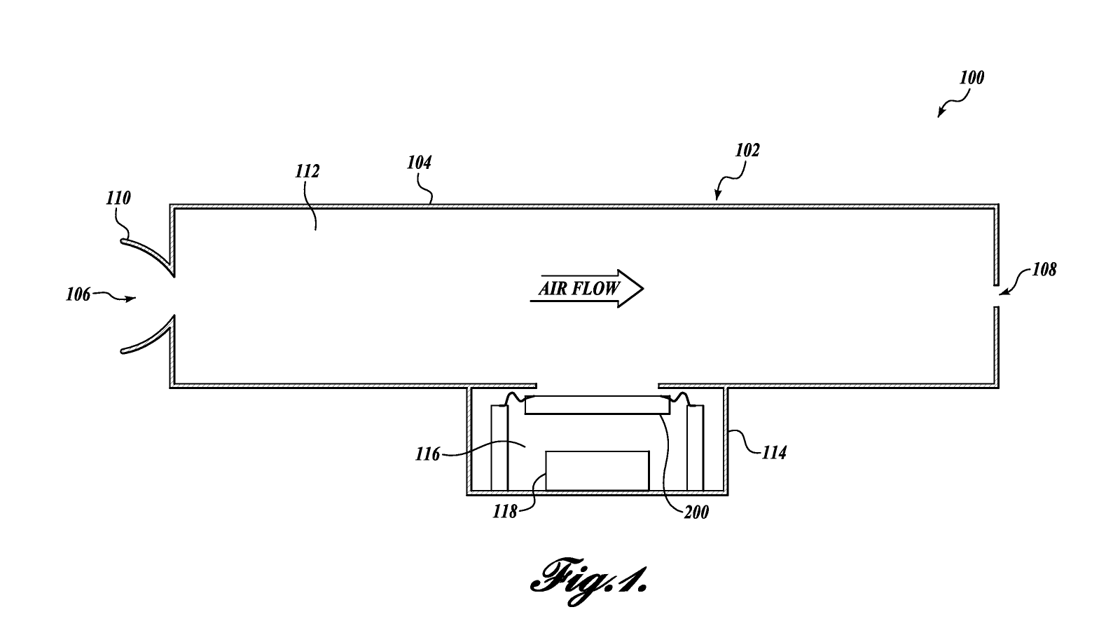

FIG. 1 shows an exemplary embodiment of an acetone sensing device 100 according to the present disclosure. The device 100 includes a breath sample collector 102 comprising an elongate body 104 with an inlet aperture 106 located at one end and an outlet aperture 108 located at the opposite end. The inlet aperture 106 has an optional mouthpiece 110 formed thereon. The mouthpiece 110 can be permanently fixed to the body 104 or may optionally be detachably coupled to the body 104 to allow for embodiments in which a disposable mouthpiece is utilized. The mouthpiece 110 optionally includes a check valve (not shown) that allows fluid to flow through the inlet aperture 106 in only one direction, i.e., into the elongate body 104. In other contemplated embodiments, the optional check valve is disposed within the elongate body 104 rather than the mouthpiece 110.

A cavity 112 is formed in a central portion of the collector 102 in fluid communication with the inlet aperture 106 and the outlet aperture 108. A sensor assembly housing 114 is positioned between the first and second ends of the elongate body and defines a sensor assembly cavity 116 in fluid communication with the cavity 112 of the elongate body 104. A sensor assembly 200 is disposed within the sensor assembly housing 114.

The sensor assembly 200 is operatively connected to a processor 118. As described in further detail below, the processor 118 receives data from the sensor assembly 200 related to sensed breath components, breath flow, sensor temperature, and other operating characteristics. In one contemplated embodiment, the processor 118 processes the data and selectively displays the information received from the sensor assembly 200 on a display (not shown) for the user. In yet another contemplated embodiment, the processor 118 stores the data locally, or makes the data available for transfer to a remote storage location or processor, such as a home computer, tablet, smart phone, etc. These and other processor functions suitable for receiving and processing diagnostic data are contemplated and should be considered within the scope of the present disclosure.

The disclosed configuration is suitable for collecting a breath sample from a user and exposing the breath sample to the sensor assembly 200 for analysis. For acetone detection, it is preferable that the analyzed breath sample be alveolar air, i.e. air from deep within the lungs. While some alveolar air is generally exhaled during the entire exhalation, in a preferred embodiment, the sample is taken from the last third of the exhalation to maximize the amount of deep-lung air in the sample. The illustrated device 100 collects and isolates alveolar air for analysis. To utilize the device 100, a user places his mouth to the mouthpiece and blows a long, continuous breath sample into the inlet aperture 106. The breath sample flows through the cavity 112 in the direction indicated by the arrow and then exits through the outlet aperture 108. The outlet aperture 108 has a reduced geometry that limits the flow of breath out of the cavity 112. In this manner, the breath sample is contained within the device 100 until after the sensor assembly 200 has analyzed the breath sample.

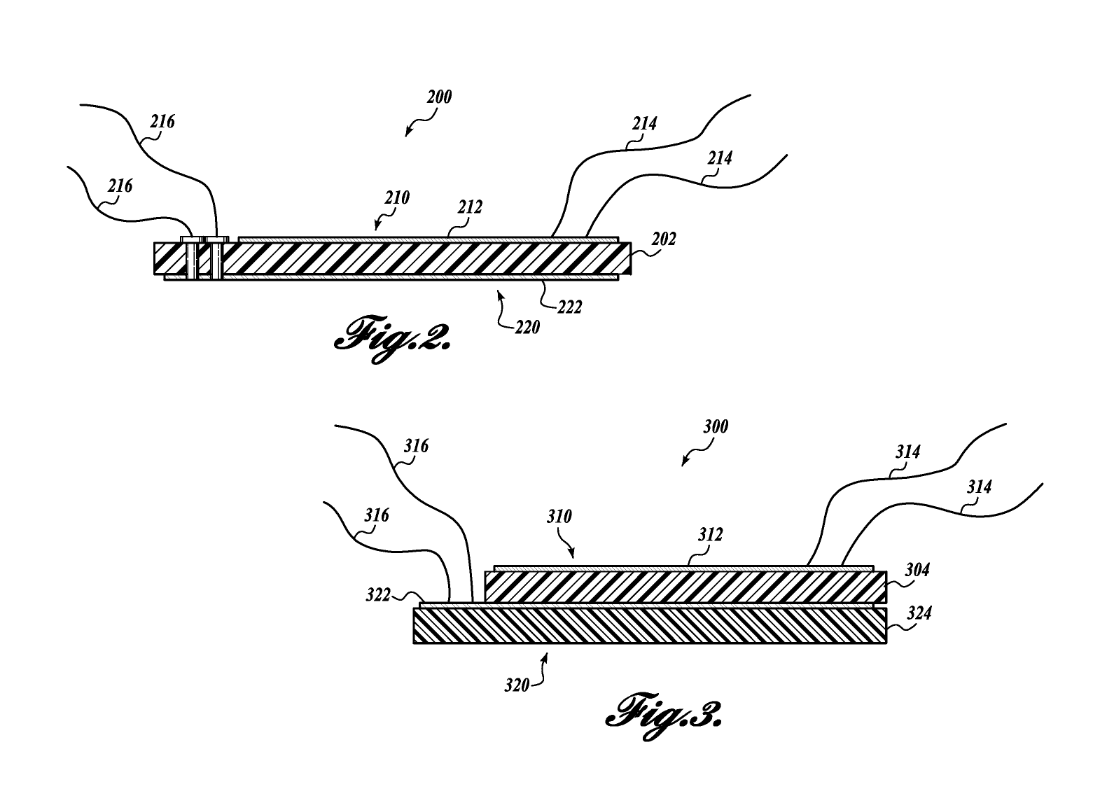

FIG. 2 shows a first exemplary embodiment of a sensor assembly 200 suitable for use in the acetone sensing device 100 of FIG. 1. The sensor assembly 200 includes a substrate 202 with an acetone sensor 210 formed on a first side and a temperature control system 220 formed on a second side. Sensor leads 214 and 216 are in electronic communication with the sensor 210 and the temperature control system 220, respectively, to provide information between the sensor assembly 200 and the processor 118. It will be appreciated that the illustrated sensor leads are exemplary only, and that any suitable configuration for operatively connecting the sensor assembly 200 to the processor 118 can be utilized. Further, the positions of the acetone sensor 210 and the temperature control system 220 on the substrate are exemplary only. In this regard, the acetone sensor 210 and the temperature control system 220 can be formed on the same side of the substrate 202 or in any other suitable locations relative to each other on the substrate.

In the illustrated embodiment, the substrate 202 is an alumina substrate with tungsten oxide (WO₃) coating 212 deposited thereon. It will be appreciated that metal oxide gas sensors are known in the art and the described WO₃ coating 212 disposed on an alumina substrate 202 is exemplary only. In this regard, other metal oxides or combination of metal oxides suitable for sensing acetone and alternate substrate materials are possible and should be considered within the scope of the present disclosure. Further, the disclosed sensor 210 is not limited to the use of metal oxide gas sensors made using any particular manufacturing method. It will also be appreciated that the substrate 202 is not limited to an aluminum oxide material, but can alternatively be formed of glass, other suitable high-temperature substrates, or a combination thereof. Exemplary metal oxide gas sensors and/or methods of forming the same are disclosed in U.S. Patent Publication Nos. 2011/0071446 and 2003/0217586, the disclosures of which are expressly incorporated herein by reference.

In the illustrated embodiment, the surface area of the sensor 210 is approximately 1 mm²; however, other embodiments are contemplated wherein the surface area of the sensor is larger or smaller than that of the illustrated embodiment. Because the surface area of the sensor is relatively small, the sensor heats-up and cools-down quickly. Metal oxide gas based sensors, such as the disclosed acetone sensor 210, typically require relatively high operational temperatures, e.g., about 300° C. The sensitivity of the sensor to a given gas is often highly dependent on the sensor temperature. Accordingly, the ability to directly monitor and control sensor temperature is advantageous.

Accurate thermal control becomes especially critical when attempting to detect gases that are present at very low concentrations such as with the acetone vapor found in breath. The presently disclosed the acetone sensing device 100 incorporates a heating element to achieve the desired sensor operating temperature, and uses of the heating element itself as the temperature measurement device. Utilizing the temperature control system 220 enables the operating temperature of the acetone sensing device 100 to be maintained within a range of approximately 300° C. to 450° C. It will be appreciated that this range is exemplary only and that the actual range of the sensor operating temperature can be modified to be suitable for a particular type of sensor. Further, the operating temperature of the sensor can be maintained within a narrower range to provide increase accuracy.

Still referring to the embodiment of FIG. 2, the temperature control system 220 is a circuit formed by depositing a platinum trace on the substrate 202. Although other materials known in the art are contemplated for the trace, platinum based resistive temperature devices (RTDs) are commonly used as temperature sensing elements due to platinum's stable resistance temperature coefficient. As used herein, RTD refers to a metal resistor with a positive temperature coefficient. In contrast to thermistors, which generally use ceramic or polymeric materials, RTDs provide more accurate readings in the temperature ranges utilized for acetone detection.

FIG. 3 shows an alternate embodiment, wherein the sensor assembly 300 includes a discrete acetone sensor 310 bonded to a discrete temperature control system 320. The acetone sensor 310 comprises a combination of a substrate 304 with a metal oxide sensor 312 disposed thereon. The temperature control system 320 is a circuit formed by depositing a platinum trace 322 on a second substrate 324. The acetone sensor 310 and the temperature control system 320 are bonded together and connected to the processor 118 by leads 314 and 316, respectively. It will be appreciated that the illustrated sensor leads are exemplary only, and that any suitable configuration for operatively connecting the sensor assembly 300 to the processor 118 can be utilized.

In order to minimize self-heating, typical RTD resistance sensing is conducted in a manner that minimizes the power applied to the RTD. Moreover, platinum is generally not used as a base material for resistive heaters due to its high cost. However, the disclosed temperature control system 220 requires a relatively small heater so that it is feasible to use the RTD itself as the resistive heating element. Integrating a resistive heating element with a temperature sensor in a single temperature control system 220 allows for significant advantages, including reduced cost, reduced sensor complexity, fewer interconnecting leads, and intimate thermal contact between heater and temperature sensor.

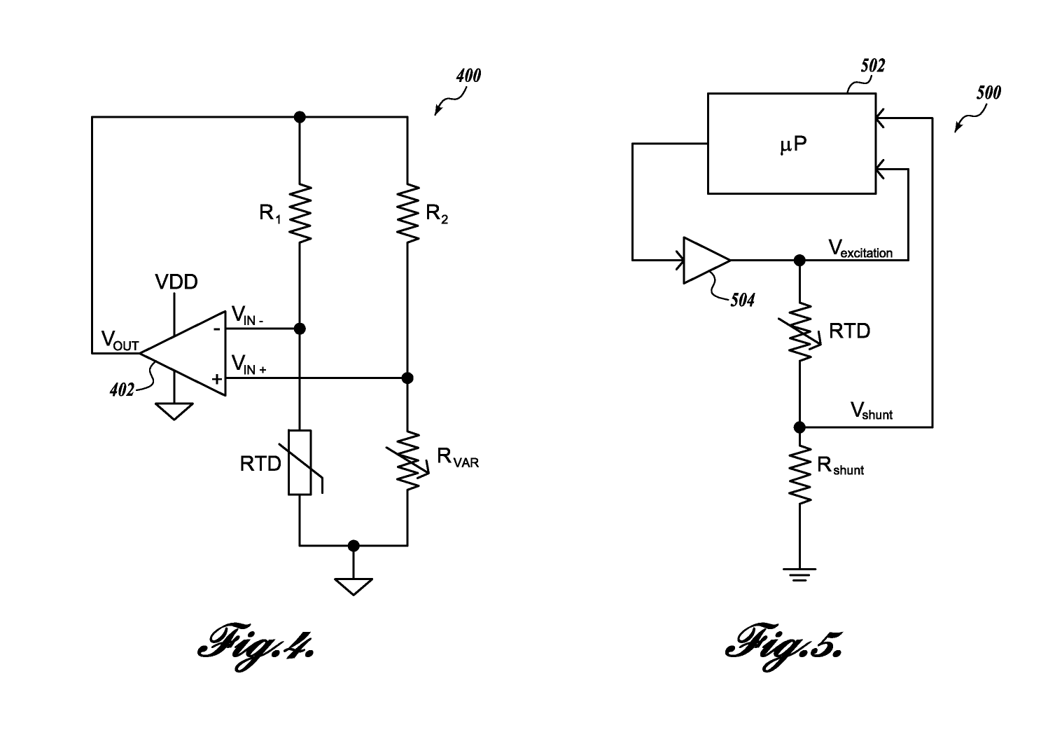

FIG. 4 shows a schematic illustration of an exemplary embodiment of a heater/temperature sensor circuit 400 suitable for use with the temperature control system 220. Generally speaking, the circuit 400 is a bridge circuit with an operational amplifier to provide closed loop control of the circuit. A first leg of the bridge includes a first resistor R₁ in series with the RTD. The second leg of the bridge includes a second resistor R₂ in series with a variable resistor R_{VAR}. The junction between R₁ and the RTD is connected to the inverting input of an operational amplifier 402, and the junction between R₂ and R_{VAR} is connected to the non-inverting input of the operational amplifier 402. The operational amplifier 402 supplies voltage to the circuit according to the difference between the voltages received from the circuit legs so that the legs of the circuit balance, as shown in equation (1).

$$\frac{{R}_{RTD}}{{R}_{1}} = \frac{{R}_{VAR}}{{R}_{2}} \qquad ( 1 )$$

The operational amplifier 402 controls the voltage so that the temperature of the RTD is such that the resistance of the RTD balances the circuit. With the R₁, R₂, and R_{VAR} having known values, the circuit is balanced when the resistance or the RTD, is at a specific value, as defined in equation (2) below.

$${R}_{RTD} = \frac{{R}_{1} {R}_{VAR}}{{R}_{2}} \qquad ( 2 )$$

The value of R_{RTD} corresponds closely to a specific RTD temperature, so that the equation is balanced when the RTD is at a predetermined temperature. In this manner, the operational amplifier 402 controls the voltage to maintain a predetermined RTD temperature.

The circuit works by the operational amplifier 402 continuously balancing its inputs, V_{IN+} and V_{IN-}, as shown below in Equation 3.

$${V}_{I N +} = {V}_{IN -} = \frac{{R}_{RTD}}{{R}_{RTD} + {R}_{1}} {V}_{OUT} = \frac{{R}_{VAR}}{{R}_{VAR} + {R}_{2}} {V}_{OUT} \qquad ( 3 )$$

When the RTD is below the temperature setpoint, its resistance is lower. Accordingly, the operational amplifier 402 input V_{IN-} is lower than V_{IN+}, which causes V_{OUT} to increase. When V_{OUT} increases, more power is delivered to the RTD, raising its temperature. Conversely, when the RTD is above the temperature setpoint, its resistance is higher. In this case, the operational amplifier 402 input V_{IN-} is higher than V_{IN+}, which causes V_{OUT} to decrease, delivering less power to the RTD and cooling it. Accordingly, the known value of R₁ can be used along with the measured values of V_{IN-} and V_{OUT} to calculate the resistance and, therefore, the temperature, of the RTD.

In addition to providing the ability to maintain a particular RTD temperature and also to sense the temperature of the RTD, the disclosed circuit 400 is also suitable for use as an anemometer. When used with the acetone sensing device 100, the heater/temperature sensor circuit 400 is subjected to a breath sample being blown past the sensor. As the breath flows past the sensor circuit 400, the effects of forced convective heat transfer requires more power to the RTD to maintain a constant temperature. Because the characteristics of the exhaled breath are known, e.g., 37° C. (body temperature for a human) with a relative humidity of ˜100%, the added power used to maintain a constant RTD temperature, which is related to cooling rate due to forced convective heat transfer, can be used to calculate the rate of flow of the breath sample as the user breathes into the acetone sensing device. Other embodiments of constant temperature anemometers are disclosed in U.S. Pat. No. 5,069,066, the disclosure of which is expressly incorporated herein.

Using the anemometer features of the disclosed temperature control system 220, it is possible to provide an acetone sensing device 100 that senses whether or not a breath sample is suitable for analysis. As previously discussed, it is preferable that the breath sample to be analyzed is from approximately the last third of a full, breath expiration. In one contemplated embodiment, the acetone sensing device 100 senses the flow rate of breath through the device and requires that the user maintain a minimum breath flow rate for a threshold amount of time before beginning acetone detection.

FIG. 5 shows a schematic illustration of a second exemplary embodiment of a heater/temperature sensor circuit 500 suitable for use as the temperature control system 220. Similar to the circuit 400 shown in FIG. 4, the circuit 500 of FIG. 5 provides closed-loop control of the temperature of an RTD, while also functioning as a temperature sensor and anemometer.

The circuit 500 includes an RTD connected in series with a shunt resistor, Rₛₕᵤₙₜ. A microprocessor 502 provides an excitation voltage (V_{excitation}) to the RTD. The junction between the microprocessor 502 and the RTD is connected to an analog input of the microprocessor 502 feeding V_{excitation} back to the microprocessor. In addition, the junction between the RTD and Rₛₕᵤₙₜ is connected to a second analog input of the microprocessor 502 feeding Vₛₕᵤₙₜ to the microprocessor. The circuit acts as a resistive divider, wherein the relationship of Vₛₕᵤₙₜ to V_{excitation} is shown in equation (4).

$${V}_{shunt} = \frac{{R}_{shunt}}{{R}_{RTD} + {R}_{shunt}} {V}_{excitation} \qquad ( 4 )$$

As previously noted, for a given RTD, a particular value R_{RTD} corresponds closely to a particular temperature of the RTD. To achieve a known R_{RTD-SETPOINT} and the corresponding target RTD temperature, the microprocessor 502 controls V_{excitation} according to equation (5).

$${V}_{excitation} = \frac{{R}_{RTD \text{-} SETPOINT} {V}_{shunt}}{{R}_{shunt}} + {V}_{shunt} \qquad ( 5 )$$

The closed-loop feedback provided through the microprocessor 502 combined with the close correlation between the resistance and the temperature of the RTD allows for the circuit 500 to also be used as a temperature sensor and as an anemometer in the manner previously described with respect to the circuit 400 of FIG. 4.

While illustrative embodiments have been illustrated and described, it will be appreciated that various changes can be made therein without departing from the spirit and scope of the invention.

The embodiments of the invention in which an exclusive property or privilege is claimed are defined as follows:

1. A breath analyzer for detecting a component in a breath sample, the analyzer comprising:
(a) a housing defining an interior cavity, the housing comprising an inlet aperture for receiving the breath sample and an outlet aperture;
(b) a sensor disposed within the cavity for sensing the component of the breath sample;
(c) an anemometer circuit associated with the sensor, the anemometer circuit measuring a rate of flow of the breath sample within the housing; and
(d) a controller operatively connected to the sensor to receive breath component information sensed by the sensor.

2. The breath analyzer of claim 1, wherein the anemometer circuit comprises a temperature control system integrally formed with the sensor, the temperature control comprising a resistive temperature device configured to heat the sensor to a predetermined temperature and to sense the temperature of the sensor

3. The breath analyzer of claim 2, wherein the sensor comprises a metal oxide deposited on a substrate.

4. The breath analyzer of claim 3, wherein the metal oxide comprises tungsten trioxide.

5. The breath analyzer of claim 3, wherein the substrate comprises aluminum oxide.

6. The breath analyzer of claim 3, wherein the temperature control system comprises a platinum metallization deposited on the substrate.

7. The breath analyzer of claim 6, wherein the metal oxide is deposited on a first side of the substrate and the platinum metallization is deposited on a second side of the substrate.

8. The breath analyzer of claim 2, wherein the temperature control system comprises a closed-loop control circuit selectively controlling the resistive temperature device.

9. The breath analyzer of claim 8, wherein the control circuit comprises a bridge circuit operably connected to an operational amplifier.

10. The breath analyzer of claim 9, wherein the bridge circuit comprises four resistors, the resistive temperature device acting as one of the resistors.

11. The breath analyzer of claim 8, wherein the control circuit comprises a processor controlling a resistive divider, resistive divider comprising the resistive temperature device connected in series with a shunt resistor.

12. The breath analyzer of claim 11, wherein the processor controls the voltage provided to the resistive divider according to a voltage drop across the resistive divider.

13. A breath analyzer for detecting a component in a breath sample, the analyzer comprising:
(a) a housing defining an interior cavity, the housing comprising an inlet aperture for receiving the breath sample and an outlet aperture;
(b) a metal oxide sensor disposed within the cavity for sensing the component of the breath sample;
(c) a temperature control system integrally formed with the sensor, the temperature control system comprising a metal resistor having a positive temperature coefficient, the metal resistor being configured to heat the sensor to a predetermined temperature and to sense the temperature of the sensor, wherein a closed-loop control circuit selectively controls the metal resistor, the metal resistor being integrally formed with the closed-loop control circuit, the closed-loop control circuit being configured to measure a rate of flow of the breath sample within the housing; and
(d) a controller operatively connected to the sensor to receive breath component information sensed by the sensor.

14. The breath analyzer of claim 13, wherein the control circuit comprises a bridge circuit operably connected to an operational amplifier.

15. The breath analyzer of claim 14, wherein the bridge circuit comprises four resistors, the metal resistor acting as one of the resistors.

16. The breath analyzer of claim 13, wherein the control circuit comprises a processor controlling a resistive divider, resistive divider comprising the metal resistor connected in series with a shunt resistor.

17. The breath analyzer of claim 16, wherein the processor controls the voltage provided to the resistive divider according to a voltage drop across the resistive divider.
